(19) United States
(12) Patent Application Publication
(10) Pub. No.: US 2013/0256230 A1
(43) Pub. Date: Oct. 3, 2013

(54) MEMBRANE SUITABLE FOR BLOOD FILTRATION

(71) Applicants: Konraad Albert Louise Hector Dullaert, Geleen (NL); Marko Dorschu, Geleen (NL); Jun Qiu, Geleen (NL); Jens Christoph Thies, Geleen (NL)

(72) Inventors: Konraad Albert Louise Hector Dullaert, Geleen (NL); Marko Dorschu, Geleen (NL); Jun Qiu, Geleen (NL); Jens Christoph Thies, Geleen (NL)

(21) Appl. No.: 13/701,321

(22) PCT Filed: May 31, 2011
(86) PCT No.: PCT/EP11/58921

§ 371 Date: Mar. 12, 2013

(30) Foreign Application Priority Data
Jun. 3, 2010 (EP) 1016-4875.6

(51) Int. Cl. B01D 71/56; G01N 33/49

(52) U.S. Cl. CPC B01D 71/56; G01N 33/491
USPC 210/650; 210/500.38; 210/483; 73/64.56

(57) ABSTRACT

The invention relates to a membrane construction comprising multiple layers wherein at least one of the layers is a nanoweb made of polymeric nanofibers, wherein the mean flow pore size of the nanoweb is in the range from 50 nm to 5 μm, wherein the number average diameter of the nanofibers is in the range from 100 to 600 nm, wherein the basis weight of the nanoweb is in the range from 1 to 20 g/m², wherein the porosity of the nanoweb is in the range from 60 to 95%, wherein at least one of the layers is a support layer and wherein the nanoweb is hydrophilic.

The invention relates to a membrane construction, a membrane cassette comprising said membrane construction, a device comprising said membrane construction or said membrane cassette and to uses thereof such as for example blood filtration, diagnostic devices, bio-separation of cell cultures and bio-fermentation.

It is one object of the invention, to provide a membrane construction that can achieve a high flux as well as a good separation. In the case when the membrane construction is used in blood filtration or in a diagnostic device the separation is mainly of blood cells from blood plasma. In case the membrane construction is used in bio-separation of cell cultures or bio-fermentation the separation is mostly of biological material from the broth. With “flux” is meant the flow of liquid through the membrane.

This object is achieved by a membrane construction comprising multiple layers wherein
a) at least one of the layers is a nanoweb made of polymeric nanofibers and
b) the mean flow pore size of the nanoweb is in the range from 50 nm to 5 μm and
c) the number average diameter of the nanofibers is in the range from 100 to 600 nm and
d) the basis weight of the nanoweb is in the range from 1 to 20 g/m² and
e) the porosity of the nanoweb is in the range from 60 to 95% and
f) at least one of the layers is a support layer and
g) the nanoweb is hydrophilic.

It has surprisingly been found that the membrane construction of the invention is very suitable for efficient separation of for example blood cells from the blood plasma. It is also very advantageous to use it in diagnostic devices, bio-separation of cell cultures and bio-fermentation.

A diagnostic device is a medical device that is intended to perform diagnoses from assays in a controlled environment outside a living organism. Here “medical devices” encompasses any device that is intended by the manufacturer to be used for the examination of specimens, including blood and tissue donations, derived from the body, solely or principally for the purpose of providing information on, for example, the physiological or pathological state. Examples of diagnostic devices are instruments, apparatus, kit, equipment, control material or system.

By using the membrane according to the present invention in a diagnostic device, it is for example possible to analyze very small amounts of blood. Diagnostic devices require an efficient method of separating plasma from a minute blood sample, often just one drop, to produce a sufficient volume of plasma to be transported through the assay portion of the device. The time allowed to complete the separation of the blood sample is also important so that the reaction underlying the analysis can be accurately completed and the results are provided in a timely manner. Preferably, in the diagnostic device, the volume of blood applied to the membrane is in the range of about 10 μl to about 30 μl, preferably less than 15 μl, which can be easily obtained from one single prick, and for the test done in the diagnostic device, 2-3 μl plasma is enough for finalization of test.

The fact that only very small amounts of blood are necessary to complete the test satisfactorily is advantageous for patients who need an analysis of their blood. For this analysis one or more blood samples are taken from the patient. It is always uncomfortable and sometimes even burdensome when these samples are taken. When a patient needs to supply more than one sample or when he needs to supply a blood sample on a regular basis (such as for example in drug monitoring or diabetes watching) it is especially advantageous when the amount of blood that has to be collected can be small. It holds in general for all patients but is especially relevant for patients having a small blood volume such as for example infants as it improves their overall health when the sample can be small compared to larger blood samples. Thus it is an important advantage when the diagnosis can be done while using less blood such as is the case with the membrane construction of the present invention.

Also, since the membrane construction of the invention provides a good flux, this membrane construction might be used for blood filtration, for example in kidney dialysis. Other applications where the speed of the transport of blood through the membrane is important also benefit from the use of the membrane construction according to the present invention.

A further advantage of the membrane construction of the invention is that it does not need to be treated with surfactants to increase hydrophilicity. Traditionally used materials are heavily loaded with surfactants to maintain the high hydrophilicity and high fluidity, and prevent hemolysis. However, the high content of surfactants results in a high percentage of surface active leachables which overwhelms the immuno-assay binding, and may result in immuno-assay interference and uneven fluidity. Additionally as there is always a risk that the surfactants that are used in coating the substrate will get detached from the substrate during the blood analysis phase, the blood in the sample can get contaminated with these surfactants. Therefore, it is an advantage when the substrate doesn't need to be coated, as the surfactants will also not be present in blood plasma resulting from the separation through the membrane construction of the invention, thereby making diagnostics easier and more accurate and reliable. When the membrane construction is used in dialysis it provides less ‘impurities’ to kidney dialysis fluids. As the membrane construction of the present invention doesn't need to be coated (but of course still can be), it is an advantage to use this membrane construction.

With membrane construction is meant a collection of layers together forming the membrane construction. With ‘multiple layers’ is meant at least two layers. Each of the layers differs in mean flow pore size and/or type of material.

It is known to the skilled person how to prepare a membrane construction comprising multiple layers of nanoweb, for example multiple layers can be made using phase inversion (e.g. as described in U.S. Pat. No. 6,045,899) or for example by spinning the nanoweb on the same place while moving a support layer or by laminating the support layer with the nanoweb. In order to attach the nanoweb to the other layers, hot laminating may be used and/or glue may for example be applied onto the support material and/or the support layer may be in a hot-melt state when the nanoweb is applied thereon.

A nanofiber web may be prepared from nanofibers using methods known to the person skilled in the art, for example via multi-nozzle electrospinning, for example as described in WO2005/073441, hereby incorporated by reference; via nozzle-free electrospinning, for example using a Nanospider™ apparatus, bubble-spinning or the like; or via electroblowing, for example as described in WO03/080905, hereby incorporated by reference.

Nanofibers may be prepared using methods known to the skilled person, for example, they may be produced using electrospinning, such as classical electrospinning or electroblowing, and sometimes also by meltblowing processes. Classical electrospinning is illustrated in U.S. Pat. No. 4,127,706, hereby incorporated by reference.

WO2008/137082 describes membranes for use in osmotically driven membrane processes. The membranes used herein consist of a non-porous material contrary to the membranes used in the construction of the present invention.

In the context of the invention, with nanoweb made of polymeric nanofibers is meant a nonwoven web comprising primarily polymeric nanofibers. Preferably the nonwoven web comprises exclusively polymeric nanofibers.

The mean flow pore size of the nanoweb is in the range 50 nm-5μm, preferably in the range from 0.1 to 4 μm, more preferably in the range of 0.5 to 3 μm.

The mean flow pore size is determined with a method using ASTM F 316. All capillary flow porometer tests were performed on a Porolux 1000 system. A capillary flow porometer measures the pore sizes and distributions of through pores in filters. In the overall methodology, a filter is wetted with a liquid. This liquid has preferably a contact angle of zero with the filter material and a known surface tension with gas at the measurement temperature. If this is the case, the pore size can be calculated using the Washburn equation: pressure (mbar)=4*surface tension (dyn/cm)/pore size diameter (μm). This is done by gradually increasing the pressure of gas over the sample in a closed container. The pressure at which an increase in gas flow is observed is then recalculated towards pore size. Typical parameters like bubble point, mean flow pore size, smallest pores and the pore size distributions are automatically calculated. The methods used for this purpose are described in ASTM F 316.

As opposed to other systems, the Porolux 1000 uses a pressure equilibrium routine. This states that between chosen boundaries the pressure and gas flow towards or through a sample have to be fully stabilized before a data point is taken as a true value. This results in very accurate measurement of the pore size diameters and very narrow but correct pore size distributions. Typically for non-woven materials, this will result in a one or two-point distribution as all openings towards these structures are interconnected throughout the complete filter. With more discrete pores like filters prepared through emulsion polymerization, through laser shooting and other methods, more broad distributions can be found.

In this series of tests stabilization routines used were a maximum deviation of 0.5% to 2% in pressure and gas flow over 1 to 2 seconds. Higher stabilization requirements were not used to exclude as much as possible the effect of dripping, evaporation of liquid through the material, and so on.

The mean flow pore size of the nanoweb may be reduced by calendering the nanoweb and/or the nanoweb in combination with the support layer. This may increase the strength of the nanoweb and/or the nanoweb/ support layer combination. Calendering is the process of passing sheet material (in this case the nanoweb) through a nip between rolls or plates.

The mean flow pore size (of the nanoweb) is influenced by a combination of the thickness of the nanoweb and the number average diameter of the nanofibers. For example, by increasing the thickness, the mean flow pore size may be reduced. By reducing the number average diameter of the nanofibers, the mean flow pore size can also be reduced.

With ‘basis weight of the nanoweb’ is meant the weight per square meter. Preferably, the basis weight of the nanoweb is in the range from 1 to 20 g/m², preferably 2-15 g/m². The basis weight is measured using ASTM D-3776, which is hereby incorporated by reference. The basis weight of the membrane construction can be determined in the same way. Preferably the basis weight of the membrane construction is in the range from 60 to 90 g/m², more preferably the basis weight is higher than 70 g/m².

The desired basis weight of the nanoweb, can be achieved by adjusting the flow rate of an electrospinning process using to spin the nanofiber and/or by adjusting the speed of the support layer onto which the nanoweb is spun.

The porosity of the nanoweb is determined as the difference between 100% and the solidity of the nanoweb. The solidity can be calculated by dividing the basis weight of the nanoweb sample in g/m², determined as described herein, by the polymer density of the polymer from which the nanofiber is made in g/cm³ and by the sample thickness in μm and multiplying by 100, i.e. solidity=(basis weight/(density*thickness)*100. Porosity=100%−% solidity. Sample thickness is determined by ASTM D-645, which method is hereby incorporated by reference, under an applied load of 50 kPa and an anvil surface area of 200 mm². Polymer density is measured as described in ISO1183-1:2004. The porosity of the membrane construction can be determined in the same way.

The porosity of the nanoweb is in the range from 60 to 95%. The porosity of the nanoweb is preferably at least 65%, more preferably at least 67%. A suitable range for the porosity of the membrane construction is at least 60 and at most 95%. Preferably the porosity is at least 65%, more preferably at least 67%. With a higher porosity, the flux through the nanoweb and the membrane construction is better. A higher porosity can also result in less loss of biomarker.

The term ‘nanofibers’, as used herein, refers to fibers having a number average diameter of at most 1000 nm (1μm). To determine the number average diameter of the fibers, ten (10) scanning electron microscopy (SEM) images at 5,000×magnification were taken of each nanofiber sample or web layer thereof. The diameter of ten (10 clearly distinguishable nanofibers was measured from each photograph and recorded, resulting in a total of one hundred (100) individual measurements. Defects were not included (i.e. lumps of nanofibers, polymer drops, intersections of nanofibers). The number average diameter, d, of the fibers was calculated from the one hundred (100) individual measurements.

A suitable range for the number average diameter of the nanofibers is from 100 to 600 nm, preferably the number average diameter of the nanofibers is at most 500, more preferably at most 400 nm. Preferably the number average diameter of the nanofibers is at least 150, more preferably at least 200 nm.

The number average diameter of the nanofiber can be varied e.g. by varying the solution concentration of the polymer solution and thus the viscosity of the polymer solution used to make the nanofibers. A generally suitable viscosity is between 200 and 1000 mPa·s. The polymer solution can contain one or more suitable solvents. The nanofiber diameter can for example be reduced by reducing the solution concentration. Another possibility to vary the diameter is to modify the process conditions such as for example the applied electrical voltage, the flow rate of the polymer solution, the choice of polymer and/ or the spinning distance. The man skilled in the art can easily, without undue experimentation or burden, determine the best set of process variables to reach the desired properties of the nanofiber.

The polymeric nanofiber may be prepared from any desired polymer material. Suitable examples of polymer materials include but are not limited to polyacetals, polyamides, polyesters, polyolefins, polyurethanes, polyacrylates, polymethacrylates, cellulose ethers and esters, polyalkylene oxides, polyalkylene sulfides, polyarylene oxides, polysulfones, modified polysulfone polymers and copolymers and mixtures thereof. Examples of materials that fall within these generic classes include poly(vinylchloride), polymethylmethacrylate and other acrylic resins, polystyrene and copolymers thereof, for example ABA type block copolymers, poly(vinylidene fluoride), poly(vinylidene chloride) polyvinylether and polyvinylalcohols.

Preferably, the polymeric nanofiber is prepared from a polyamide chosen from the group of aromatic polyamides, semi-aromatic polyamides, aliphatic polyamides, mixtures and copolyamides of semi-aromatic and/or aromatic and/or aliphatic polyamides. More preferably the polymeric nanofiber is prepared from the group of aliphatic polyamides, mixtures and copolyamides thereof. Aliphatic polyamides are preferred over aromatic and semi-aromatic polyamides when used for electrospinning of the nanofibers since aromatic and semi-aromatic polyamides usually require more hazardous solvents and are less hydrophilic than the aliphatic polymers. The polyamides may be crystalline, semi-crystalline or amorphous. Preferably, the polymeric nanofiber is prepared from a semi-crystalline polyamide, more preferably the polymeric nanofiber is prepared from a semi-crystalline aliphatic polyamide.

As used herein, the term polyamide encompasses for example polyamides comprising proteins such as for example silk or keratin as well as modified polyamides, such as for example hindered phenol end capped polyamides.

Examples of aromatic polyamides, also known as polyaramides, are poly-p-phenylene terephthalamide (PPTA, commercially available as for example Kevlar™, Twaron™ or Technora™) or poly-p-phenylene isophthalamide (PPIA, commercially available as Nomex™)

Examples of semi-aromatic polyamides, include terephthalic acid (T) based polyamides, for example polyamide 4,T, polyamide 6,T/6,6, polyamide 9,T, polyamide 6,T/6,I (a copolyamide based on hexamethylene diamine and isophthalic acid and terephthalic acid) or PAMXD,6 (a polyamide based on 1,3-xylylendiamine and adipic acid), PAMXD,T (a polyamide based on 1,3-xylylendiamine and terephthalic acid) or copolyamides thereof.

Examples of suitable aliphatic polyamides are polyamide-2 (polyglycine), polyamide-3, polyamide-4, polyamide-5, polyamide-6, polyamide-2,6, polyamide-2,8, polyamide-6,6, polyamide-4,6, polyamide-4,10, or polyamide-6,10 or copolyamides and/or mixtures thereof, such as for example the copolyamides polyamide 6/6,6, polyamide 4,6/6;

Preferably, the polymeric nanofiber is made from alcohol soluble polyamides. Such alcohol soluble polymers are for example commercially available from BASF under the name Ultramid®, for example Ultramid®1 C. This material is an aliphatic block-copolyamide.

Preferred thermoplastic polyamides include but are not limited to polyamide-6; polyamide-6,6; polyamide-4,6; polyamide-4,10; polyamide-6,10; copolyamides and/or mixtures thereof, more preferably polyamide-6, polyamide-6,6, polyamide-4,6, copolyamides and/or mixtures thereof. Most preferably polyamide-4,6, copolyamides and/or mixtures thereof are used. Polyamide-4,6 is a class of polyamides commercially available under the trademark Stanyl™ from DSM, the Netherlands. If the nanoweb is made from nanofibers made from these preferred thermoplastic polyamides, the nanoweb has a high hydrophilicity, high thermal stability, improved water flux compared to less hydrophilic polymers and a high (tensile) strength.

Preferably, the polyamide has a carbon/nitrogen (C/N) ratio of at most 9, more preferably, the polyamide has a C/N ratio in the range of from 4-8. When the C/N-ratio is in this preferred range, the hydrophilicity is most advantageous.

A more hydrophilic polymeric material has a better wettability with polar liquids, such as for example blood and water, which are generally used in the fields where the membrane construction of the present invention can advantageously be used. Wettability can be determined by a simple water deposition test. 10 μl demineralized water is dropped onto the membrane surface with a pipette. In this embodiment, where water (a polar liquid) is used, a high wettability means that the water almost instantaneously penetrates the membrane and spreads over the surface. No water droplets are formed on the surface. A membrane material that has a high wettability with water is a hydrophilic material. Surprisingly it was found that the use of polymeric materials that have a higher hydrophilicity results in less absorption of proteins when the membrane is used in a blood filtration application.

Tensile strength can be measured on an extensometer (MTS QUEST™ 5 at a constant rate of elongation of 2 inches per minute. Samples are cut to a size of 1 inch by 8 inches, being longer in the direction of loading. The gage length of the samples was 6 inches and the starting width of samples was 1 inch. The tensile strength is defined as the maximum load supported by a sample piece of the nanoweb divided by its cross-sectional area (A=width×thickness). Samples are tested in both the X (length) and the Y (width) direction.

Water flux is the amount of clean water (in liter) that passes through the nanoweb, the membrane construction or the support layer, per hour at 1 bar per m² of the material through which it passes (respectively the nanoweb, the membrane construction or the support layer).

Thermal stability of a material is indirectly determined via its tensile strength, by heating a sample of the material to be tested (e.g. the nanoweb, the membrane construction or the support layer) in an oven at an elevated temperature and measuring the tensile strength of the sample over time. A material that retains its tensile strength up to a higher temperature has a higher thermal stability.

In the polymer solution comprising the polymeric material of choice used to prepare the nanofibers, additives may be present. Suitable additives include but are not limited to: surface tension agents or surfactants, for example perfluorinated acridine, crosslinking agents, viscosity modifiers, for example hyperbranched polymers such as hydroxylfunctional hyperbranched polyester amide polymers as described in WO1999/016810, carboxyfunctional hyperbranched polyester amide polymers as described in WO2000/056804, dialkylamide functional hyperbranched polyester amide polymers as described in WO2000/058388, ethoxyfunctional hyperbranched polyester amide polymers as described in WO2003/037959, heterofunctionalized hyperbranched polyester amides as described in WO2007/098889 or secondary amide hyperbranched polyester amides as described in WO2007/144189, electrolytes, antimicrobial additives, adhesion improvers, for example maleic acid anhydride grafted rubber or other addtives to improve adhesion with a polypropylene or polyethylene terephthalate substrate, nanoparticles, for example nanotubes or nanoclays, and so on.

Examples of electrolytes include water soluble metal salts, for example metal alkali metal salts, earth alkali metal salts and zinc salts, LiCl, HCOOK (potassium formate), CaCl₂, ZnCl₂, KI₃, NaI₃. Preferably, an electrolyte is present in an amount in the range of from 0 to 2 wt % relative to the total weight of the polymer solution. The water soluble salt may be extracted with water from the nanofibers produced, thereby obtaining microporous nanofibers.

In certain application fields of the membrane construction, it is an advantage when as little as possible additives are present in the polymer of which the nanofibers are made. These fields are for example blood filtration and/or diagnostic devices. Preferably no additives are present as when no additives are present in the nanofiber, there is no chance that the streams that pass through the membrane become contaminated by additives that leach out from the polymer.

The weight average molecular weight (Mw) of the thermoplastic polymer is preferably at least 10,000, for example at least 25,000 and/or at most 50,000 , for example at most 40,000, for example at most 35,000 g/mol. These numbers particularly apply also to the preferred polyamide. The advantage, when using polymers with their molecular weight in the indicated range, is that the process of producing nanofibers from these polymers can run at an advantageously high speed, while still producing fibers with an appropriate strength.

Polyvinylalcohol (PVA), which has the general formula (C₂H₄O)ₙ preferably has an weight average molecular weight (Mw) of at least 10,000, for example at least 25,000 and/or at most 50,000, for example at most 40,000, for example at most 35,000 g/mol. The density of the polyvinylalcohol is preferably in the range of from 1.19 to 1.31 g/cm³. Since PVA is soluble in water, it is possible to use a solution of PVA in water for electrospinning of the nanofibers. This offers the possibility to spin a nanoweb free of solvent contaminants without needing a drying or other step to remove the solvent. This is especially advantageous when the nanoweb is used in a membrane construction according to the invention for blood filtration. Furthermore, the nanoweb prepared from nanofibers made from PVA may have a high wettability with a non-harmful solvent, namely water.

A generally applied process for the preparation of nanofibers using an electrospinning process comprises the steps of:
applying a high voltage between a spinneret comprising a series of spinning nozzles and a collector, or between a separate electrode and a collector,
feeding a stream of polymer solution comprising a polymer and a solvent to the spinneret,
whereby the polymeric solution exits from the spinneret through the spinning nozzles and transforms under the influence of the high voltage into charged jet streams,
whereby the jet stream is being deposited on or taken up by the collector or a support layer,
whereby the polymer in the jet stream solidifies prior to or while being deposited on or taken up by the collector or the support layer whereby the nanofibers are formed.

After preparation of the nanofibers, the nanofibers may be post-stretched, washed, dried, cured, annealed and/or post condensed. It may be advantageous to dry the nanofibers to remove residual solvents which may interfere with the analysis of the blood plasma obtained after filtration using the membrane construction of the invention.

A detailed description on how polyamide-46 nanofibers may be prepared is for example given by Huang, C. et al., ‘Electrospun polymer nanofibers with small diameters’, Nanotechnology, vol. 17 (2006), pp 2558-2563.

Crystalline polymers have a melt temperature (Tₘ) and do not have a glass transition temperature (T_{g}). Semi-crystalline polymers have both a melt temperature (Tₘ) and a glass transition temperature (T_{g}), whereas amorphous polymers only have a glass transition temperature (T_{g}) and do not have a melt temperature (Tₘ). Glass transition temperature (T_{g}) measurements (inflection point) and melting temperature (Tₘ) measurements are carried out via differential scanning calorimetry (DSC) on a Mettler Toledo, TA DSC821, in N₂ atmosphere and at a heating rate of 5° C./min. Melting temperature (Tₘ) and glass transition temperature (T_{g}) were determined using the second heating curve.

The membrane construction of the invention comprises at least one support layer. The support layer may be any substrate on which the nanoweb can be added, for example a non-woven cloth, any fibrous substrate, or a filter or membrane layer, for example a microporous membrane. A microporous layer is a layer wherein the mean flow pore size is at least 5 μm. The mean flow pore size of the support layer should be larger than the mean flow pore size of the nanoweb. For example, the mean flow pore size of the support layer may range from more than 5 μm to 100 μm. Preferably, the mean flow pore size of the support layer is at least 25 μm, more preferably at least 50 μm.

In order to keep the amount of dead volume limited, the thickness of the support layer is preferably not more than 400 μm, more preferably less than 300 μm. The thickness is generally at least 1 μm, preferably at least 10 μm. A higher value for the dead volume is disadvantageous as more fluids, such as for example blood are retained in the membrane construction, thus less plasma is generated and more blood is required to obtain the same volume of plasma.

The porosity of the support layer is suitably at least 50%, preferably at least 60%, more preferably at least 70%, even more preferably at least 80% but most preferably at least 90%. The porosity of the support layer can be determined in the same way as described for the nanoweb and the membrane construction.

Water flux is the amount of clean water (in liter) that passes through the nanoweb, the membrane construction or the support layer, per hour at 1 bar per m² of the material through which it passes (respectively the nanoweb, the membrane construction or the support layer). The water flux of the support layer is preferably at least 10,000, more preferably at least 20,000, for example at least 30,0001.h⁻¹.m⁻²if measured at atmospheric pressure (1 bar).

In a special embodiment, the membrane construction comprises more than one support layer, wherein the support layers form a gradient pore structure. With ‘gradient pore structure’ is meant that the mean flow pore size in the membrane construction changes in successive layers of the membrane construction. In a preferred embodiment, the mean pore size diminishes in successive layers so the mean flow pore size is largest at the side of the membrane construction where the first contact occurs between the liquid and the membrane construction and is smallest at the side where most of the liquid leaves the membrane construction. The side of the membrane construction where the first contact occurs between the liquid and the membrane construction will here and hereinafter be referred to as the top side. The side where most of the liquid leaves the membrane construction will here and hereinafter be referred to as the down side. Thus a preferred embodiment is a membrane construction wherein the layer at the topside has the largest mean flow pore size and the layer at the down side has the smallest mean flow pore size. Optionally an intermediate layer is present with an intermediate mean flow pore size.

In another embodiment of the invention, the membrane construction comprises only one support layer and only one layer of nanoweb. In a preferred embodiment the support layer is at the top side of the membrane construction and the nanoweb is at the down side of the construction.

The support layer is preferably hydrophilic; the support layer may be prepared from hydrophilic materials or if the support layer is prepared from hydrophobic material, the support layer may be coated with a hydrophilic coating as described herein. Preferably both the support material and the nanoweb are hydrophilic.

Examples of suitable support materials are microporous membranes, fibrous substrates, woven and non-woven cloths or any combination thereof. The latter include for example a meltblown nonwoven cloth, needle-punched or spunlaced nonwoven cloth and knitted cloth. Suitable examples of fibrous substrates include, paper and any fibrous substrate selected from the group of materials comprising glass, silica, metals, ceramic, silicon carbide, carbon, boron, natural fibers such as for example cotton, wool hemp or flax, synthetic fibers such as for example viscose or cellulosic fibers or fibers made for example from polyester, polyamide, polyacryl, polyolefine, synthetic rubber, polyvinylalcohol, aramide and chlorofibers and/ or fluorofibers or any combination thereof.

If a microporous membrane is used as the at least one support layer, the membrane may be prepared from any polymer, for example polyamide, preferably an aliphatic polyamide, for example polyamide-6, polyamide-46, a copolymer or a mixture thereof. Further suitable examples of polymers are a polyolefin or a halogenated vinyl polymer. A preferred halogenated vinyl polymer is polytetrafluoroethylene (PTFE). A preferred polyolefin is a polyethylene (PE), more preferably an ultra high molecular weight polyethylene (UHMWPE), which has a weight average molecular weight (Mw) of at least 0.5*10⁶ g/mol. A microporous membrane made from UHMWPE is for example available from Lydall, the Netherlands under the name Solupor™. Depending on the nature of the material used it may be advantageous for certain applications to coat the material with a suitable coating, for example a hydrophilic coating when the material has a hydrophobic nature.

The amount of polyolefin or halogenated vinyl polymer present in the microporous membrane is for example at least 20 wt %, for example at least 50 wt % relative to the total weight of the microporous membrane.

Microporous membranes may be prepared using methods known to the skilled person. For example in U.S. Pat. No. 3,876,738 it is described that microporous films may be produced by a process of quenching a polymer solution cast in a quench bath containing a non-solvent system for the polymer to form micropores in the resulting polymer film. For example, U.S. Pat. No. 5,693,231 describes a process for the preparation of microporous polymeric membranes and U.S. Pat. No. 5,264,165 describes a process for the preparation of a polyamide-46 microporous membrane.

The basic weight of the support layer is in principle not critical and may for example be in the range of from 1 to 300 g/m².

Preferably, the nanoweb and the one or more support layers are in contact with one another as this may provide mechanical support and/or a reduced amount of so-called ‘dead volume’, that is the volume where the liquid to be separated stays inside the membrane construction rather than flowing through.

The membrane construction may comprise further layers besides the nanoweb and the support layer. These layers may be layers to increase the separation of the components to be separated and/or to increase the tensile strength of the membrane construction. For example, the membrane construction may further comprise, a ‘functional’ membrane layer, a further nanoweb layer, and/ or a textile layer. Such a textile layer is preferably in contact with the support layer if a microporous support layer is present in the membrane construction according to the invention. The textile layer may also be the support layer if no microporous support layer is present in the membrane construction of the invention. In that case, the textile layer and the nanoweb are preferably in contact with one another. The textile layer can for example be any non-woven support or any fibrous substrate as described above. An advantageous membrane construction is a construction comprising three layers with on top a non-woven layer made out of polyamide, a second layer made out of polyamide and a third layer made out of a polyamide nanoweb. The thicknesses of the support layers are preferably about 75 μm and 20 μm. An advantage of this construction is that it is able to filter a high amount of blood or other bio-material containing fluid streams.

In case a nanoweb is spun directly onto a support surface having a large mean flow pore size, multiple nanowebs forming a nanofiber gradient may be used. WO2008/142023 A2 describes for example, how to spin a multiple layer gradient nanoweb. In the present invention a two layer nanoweb can be prepared, wherein for example a top layer is prepared from nanofibers having a number average diameter in the range of from 400 to 600 nm and the other, lower, layer can be prepared from nanofibers having a number average diameter in the range of from 100 to 390 nm.

As defined herein, two layers are preferably ‘in contact with one another’ by being bonded, adhered or laminated together.

In a special embodiment, at least one of the layers of the membrane construction is coated. With ‘coated’ is meant that the at least one layer is contacted with a coating solution, such that the coating solution impregnates the layer. So, for example the nanoweb layer and/or the support layer and/or any other further layer of the membrane construction may be coated.

The nanoweb and/or the microporous support can be coated with a non-biofouling coating by immersion of the nanoweb and/or the microporous support in a non-biofouling solution as described herein and in Holmes, P. F. et al., Journal of Biomedical Materials Research Part A, Surface-modified nanoparticles as a new, versatile, and mechanically robust nonadhesive coating: Suppression of protein adsorption and bacterial adhesion, volume 91, Issue 3, Date: 1 December 2009, Pages: 824-833.

Examples of coating solutions include antifouling coating solutions, for example antibiofouling coating solutions such as for example described in WO2006/016800. WO2006/016800 discloses a coating solution comprising particles that are grafted with reactive groups and hydrophilic polymer chains. The particles are preferably inorganic particles having an average smallest diameter of less than 10 μm, for example SiO₂, TiO₂, ZnO₂, SnO₂, Am—SnO₂, ZrO₂, Sb—SnO₂, Al₂O₃, Au or Ag particles. The hydrophilic polymer chain may comprise monomer units of ethyleneoxide, (meth)acrylic acid, (meth)acrylamide, vinylpyrrolidone, 2-hydroxyethyl(meth)acrylate, phosphorylcholine, glycidyl(meth)acrylate or saccharides.

Other antibiofouling coating solutions are for example described in WO2010/049535. WO2010/049535 discloses a coating composition comprising nanoparticles grafted with a reactive group and hydrophilic polymer chains in a solvent having a surface tension at 25° C. of below 40 mN/m. The reactive group may be selected from the group of acrylates, methacrylates, epoxy, vinyl ethers, allyl ethers, styrenics, or combinations thereof. The hydrophilic polymer chain may comprise monomer units of ethylenoxide, (meth)acrylic acid, (meth)acrylamide, vinylpyrrolidone, 2-hydroxyethyl(meth)acrylate, phosphorylcholine, glycidyl(meth)acrylate or saccharides. The nanoparticles may comprise SiO₂. The coating composition may comprise a UV-photoinitiator and may comprise a solvent selected from the group of water, methanol, ethanol, isopropanol, n-propanol, butanol, isobutanol, acetone, methylether ketone, methylisobutyl ketone, isophorone, amyl acetate, butyl acetate, ethyl acetate, butylglycol acetate, butyl glycol, ethyl glycol, 2-nitropropane, and combinations thereof.

In a preferred embodiment at least one of the layers of the membrane construction is coated with an antibiofouling coating. By coating at least one of the layers of the membrane construction with an antibiofouling coating, protein recovery in the blood plasma is increased. If the membrane construction is used for diagnostics, this will enhance the analysis resolution. If the membrane construction is used for dialysis, the efficiency of the dialysis will be increased.

Alternatively, at least one of the layers of the membrane construction, preferably the support layer, preferably the microporous membrane may be coated using a polymer coating solution, for example a solution comprising a polymer chosen from the group consisting of polyesters, polyamides, for example polyamides as described herein, for example polyamide-46, polyurea, polyurethanes, or a combination or blend or an elastomeric copolymer derivative thereof. A description on how to impregnate a membrane layer is for example given in WO2009/063067.

An advantage of using a coating solution comprising polyamide-46 is that the thermal stability of the membrane construction is increased. If a polyamide-46 coating solution is used to coat the support layer, the support layer shows improved adhesion to the nanoweb, making techniques such as hot-melt or applying glue to the support layer superfluous.

The use of a hydrophilic polymer, such as polyamide-46 in a polymer coating solution offers the opportunity to transform a hydrophobic nanofiber or hydrophobic layer into a hydrophilic nanofiber respectively a hydrophilic layer. As discussed above, the higher the hydrophilicity of the membrane construction, the better the wettability and water flux of the membrane construction.

The layer to be coated can for example be the support layer and/or the nanoweb made of nanofibers and/or the textile layer and/or any other further layer.

In one embodiment, the invention relates to a membrane construction comprising as a top layer a layer of a microporous membrane of ultra high molecular weight polyethylene or (extended) polytetrafluoroethylene, wherein the microporous membrane has been coated with polyamide-46 and/or with an anti(bio)fouling coating and a down side layer of a nanoweb of polyamide-46 nanofibers, wherein the nanoweb is preferably coated with an antibiofouling coating as described above.

In another embodiment, the invention relates to a membrane construction comprising as a top layer a non-woven support layer, wherein the support layer may be coated with an anti(bio)fouling coating and as down side layer a nanoweb of polyamide-46 nanofibers, wherein the nanoweb is preferably coated with an antibiofouling coating as described above.

In another embodiment, the invention relates to a membrane construction comprising as a down side layer a nanoweb of polyamide-4,6 nanofibers, wherein the nanoweb is preferably coated with an antibiofouling coating as described above and as a top-layer a microporous membrane of hydrophilic polyamide, wherein the microporous membrane may be coated with an anti(bio)fouling coating.

In one aspect, the invention relates to a membrane cassette comprising the membrane construction of the invention. With membrane cassette is meant a construction (housing) containing one or more membrane constructions of the invention.

In another aspect, the invention relates to a device comprising the membrane construction of or the membrane cassette of the invention. Such devices may be devices used in plasma and serum separation in for example diagnostics; pre-analytical systems, such as blood collection devices, for example tubes and capillaries or biosensors. Also, such devices may be devices wherein filters are used for extra corporal circulation circuits, such as for example in bypass surgery, blood oxygenation and aphaeresis.

In kidney dialysis, the membrane construction of the invention is preferably used in combination with a back-flush mechanism. A back-flush mechanism has the advantage that fouling of the membrane construction is reduced, thereby making it possible to maintain high flux during longer periods in time.

The invention also relates to the use of the membrane construction, the membrane cassette or of the device of the invention for blood filtration or for diagnostics.

The invention also relates to the use of the membrane construction, the membrane cassette or of the device of the invention for the use of any one of the following applications: molecular separations and filtration, like gas/gas filtration, hot gas filtration, particle filtration, liquid filtration such as micro filtration, ultra filtration, nano filtration, reverse osmosis; waste water purification, oil and fuel filtration; controlled release applications including pharmaceutical and nutraceutical components; pertraction, pervaporation and contactor applications; immobilization of enzymes, and humidifiers, drug delivery; (industrial) wipes, surgical gowns and drapes, wound dressing, tissue engineering, protective clothing, catalyst supports, and various coatings.

The invention will now be elucidated with the following examples, without however being limited thereto.

### EXAMPLES

A nanoweb was prepared from polyamide-4,6, that was prepared via standard polymerisation techniques. The nanoweb was prepared using a solution of the polyamide-4,6 in a mixture of formic and acetic acid using electrospinning as described herein. The mixture consisted of 40 wt % formic acid and 60 wt % acetic acid. The formic acid was obtained from Merck (Proanalyse, 98 -100%). The acetic acid was also obtained from Merck (99+%).

The support on which the nanoweb was spun was Novatexx 2597. Novatexx 2597 is a non-woven support material that is commercially available from Freudenberg Filtration Technologies KG. It is a support material that is based on a blend of polyamide-6 and polyamide-6,6.

Of the nanoweb made out of PA-4,6 and the support used, the wettability was determined. All show immediate wetting. Comparative Example A was also tested on its hydrophilicity. It appeared that both sides of the filter differ in hydrophilicity, with the side with the largest pores having the highest hydrophilicity. On the side with the smallest pores (the side with the non-shiny surface) the water droplet stayed for a while and only slowly started to spread, indicative for a less hydrophilic nature.

### Experiments

To test the performance of the membrane construction according to the invention, the membrane construction and a prior art blood separation filter (comparative) were used in a blood separation test. In the blood separation test, 20 μl of fresh blood from healthy volunteers was deposited on the top of the membrane construction and on the top of the comparative filter. The comparative filter was a commercially available filter from Pall Corporation. The filter is sold as a Pall Vivid GF filter.

In the blood separation test it was determined how quickly the blood moved through the membrane construction (‘blood vertical wicking’) and how quickly the blood spread on top of the membrane construction (‘blood lateral wicking’). Furthermore, it was determined in how far the blood particles on top of the membrane construction or comparative filter spread in lateral direction; this was also determined by visual inspection. (The results could be: slightly reddish, meaning that the blood cells spread in lateral direction, whereas yellowish means that the blood cells hardly spread in lateral direction).

Additionally it was determined if the blood plasma that went through the membrane contained a lot of blood particles or not, which is indicative for the separation performance. This separation performance was determined by visual inspection. When the plasma that passed through the membrane construction or comparative filter was clean, it meant that the blood plasma contained hardly any blood cells.

Additionally of some of the examples and of the comparative example it was determined what the coagulation activating potential for the materials used was. This was done by means of thrombin generation. Punched parts of the filter material (round, diameter 5 mm) were incubated with 3.2%(w/v) citrated platelet poor plasma (PPP) with a low basal contact activation. The filter disks were incubated with 180 μl PPP in a 96 well plate for 15 and 30 minutes while shaking at room temperature. Immediately after incubation, two times 80 μl of each sample were transferred to a new 96 well plate for thrombin generation.

Thrombin generation in human platelet-poor plasma in the absence or presence of a filter disc was measured by means of the CAT method (Thrombinoscope BV), which employs a low affinity fluorogenic substrate for thrombin (Z-Gly-Gly-Arg-AMC) to continuously monitor thrombin activity in clotting plasma. Measurements were conducted in 80 μl human platelet-poor normal pooled plasma in a total volume of 120 μl. To the 80 μl plasma sample, 20 μl of MP-reagent (0 pM TF, 24 μM phospholipids at 20:20:60 mol % PS:PE:PC were added. This MP-reagent can be commercially obtained from Thrombinoscope B.V., the Netherlands. After 10 minutes incubation at 37° C., 20 μl FluCa (2.5 mM fluorogenic substrate, 87 mM Calcium chloride was added to start recording of the thrombin generation.

In order to correct for inner-filter effects and substrate consumption, each thrombin generation measurement was calibrated against the fluorescence curve obtained in a sample from the same plasma (80 μl), added with a fixed amount of thrombin-α2-macroglobulin complex (20 μl Thrombin Calibrator, Thrombinoscope BV) and 20 μl FluCa (2.5 mM fluorogenic substrate, 100 mM Clacium chloride). Fluorescence was read in a Fluoroskan Ascent reader (Thermo Labsystems OY, Helsinki Finland) equipped wih a 390/460 filter set and thrombin generation curves were calculated with the Thrombinoscope software (Thrombinoscope BV).

Additionally of some of the examples and of the comparative example it was determined whether the materials used adsorbed proteins in the blood. Punched parts of the filter material (round, diameter 5 mm) were incubated with 75 times diluted Normal Pool PPP 2011 (NP11). The reference blood sample NP11 was obtained in a method known to the man skilled in the art, see for example Thrombosis and

Haemostasis, 2008, 100(2) (August), pg 362-364, herewith incorporated by reference . Incubation was done for 60 minutes at room temperature while shaking. Hereafter the total protein content of the incubated plasma was assessed. Total protein was assessed by means of the DC™ (detergent compatible) protein assay (Bio-Rad), which is a colorimetric assay for protein concentration following detergent solubilization. The DC protein assay is measured at 650-750 nm with a standard laboratory spectrophotometer or microplate reader.

The results are shown in the following Tables and Figures.

### Explanation to the Tables

Table 1 gives a description of the materials used in the membrane construction (both according to the invention and comparative Examples),
Table 2 describes properties of the membrane construction and
Table 3 describes the results of the blood separation test.
Comparative Example A is a commercially available Pall Vivid GF filter,
Comparative Example B is a non-woven support material: Novatexx 2597, that is commercially available from Freudenberg Filtration Technologies KG.
Example 1, 2, 3 and 4 are all PA-4,6 nanoweb membrane constructions that are electrospun onto the Novatexx 2597 non-woven support of Comparative Example B. The Examples 1-4 differ in mean flow pore size. For further details see Table 1.

The amount of leachables was determined by weighting the sample before and after the sample was washed with ethanol, followed by drying in air.

### Results

From the results in Table 1 it can clearly be derived that the use of materials according to the invention (Examples 1-4) lead to less leachables than the prior art materials (Comparative Example 1). Further it can de concluded from Table 2 that the dead volume of membrane constructions according to the invention is much less than from the prior art material. From Table 3 it can be concluded that with the membrane construction according to the invention the blood separation times are much shorter. Additionally, the generated plasma volume per μl of blood is higher with the membranes according to the invention compared to prior art materials.

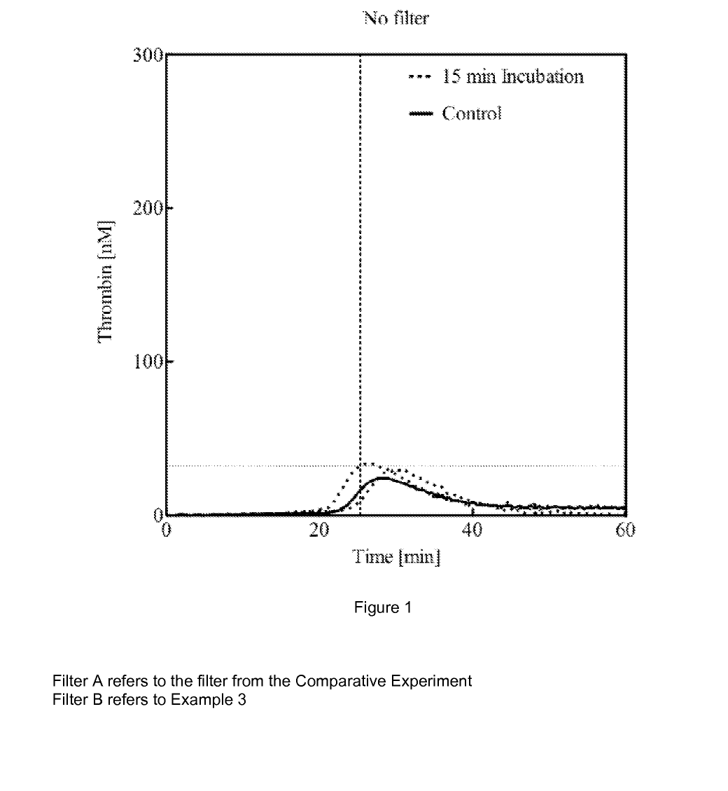

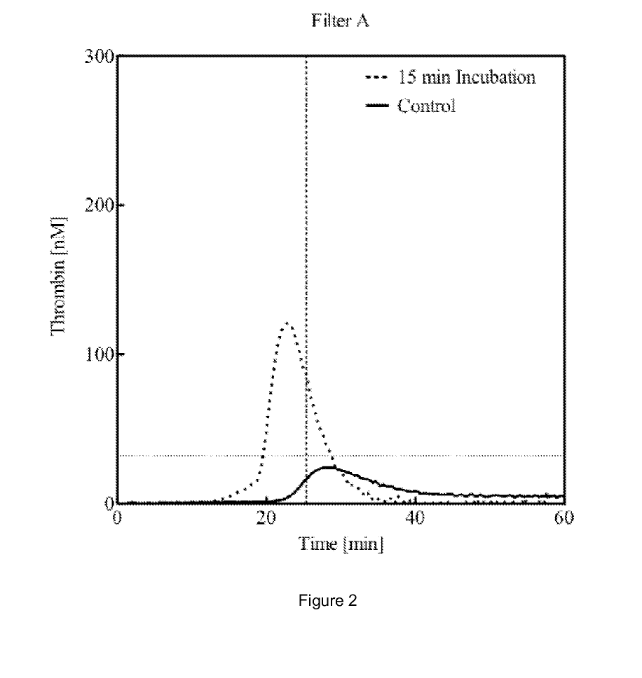

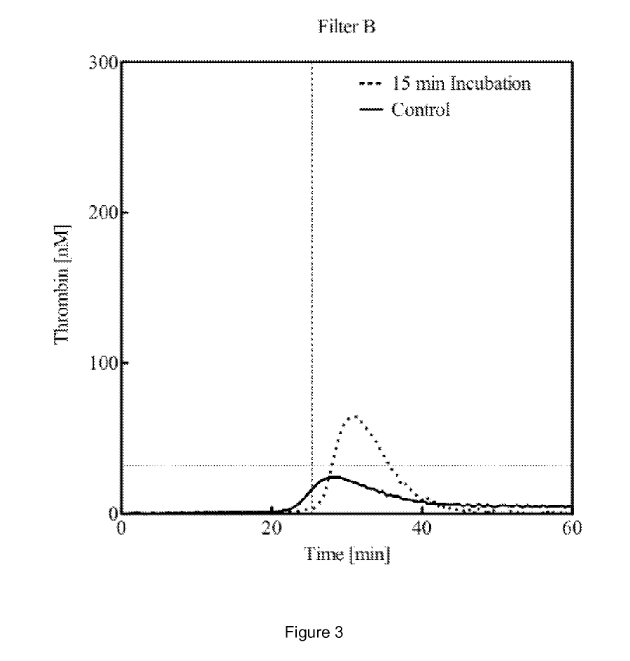

It can be clearly observed that the use of a membrane construction according to the invention (FIG. 3), leads to less coagulation activating potential than when a filter from the prior art is used (FIG. 2). FIG. 1 is incorporated for reference purposes only and shows the results of the measurements when no filter is used during the analysis.

In the test to determine the amount of proteins that were absorbed onto the membrane construction it appeared that the membrane construction according to the present invention absorbed no detectable amounts of proteins from the blood.

TABLE 1
Material description

| | | Comparative | Comparative | | | | |
|---|---|---|---|---|---|---|---|
| Features | Unit | Example A | Example B | Example 1 | Example 2 | Example 3 | Example 4 |
| Material | | Polyether | PA6/PA66 | PA46 | PA46 | PA46 | PA46 |
| | | Sulfone | | | | | |
| Density | g/m³ | 1.24 | 1.13 | 1.18 | 1.18 | 1.18 | 1.18 |
| Process | | phase inversion | spun bound | electro- | electro- | electro- | electro- |
| | | | | spinning | spinning | spinning | spinning |
| construction | | asymmetric | symmetric | asymmetric | asymmetric | asymmetric | asymmetric |
| Support layer | | non | non | Example B | Example B | Example B | Example B |
| Surfactants/coating | | PVP | Non | Non | Non | Non | non |
| Weigth loss | % | <2.5 | 0.5 | 1 | 0.8 | 0.8 | 0.8 |
| leachable | | | | | | | |
| Wicking in lateral | Sec. | N/A | N/A | N/A | N/A | N/A | N/A |
| Wicking in vertical | Sec. | >10 | <3 | <3 | <3 | <3 | <3 |

TABLE 2
Blood separation membrane properties

| | | Comparative | Comparative | | | | |
|---|---|---|---|---|---|---|---|
| Feature | Unit | Example A | Example B | Example 1 | Example 2 | Example 3 | Example 4 |
| Mean flow | μm | 1.9 | 39 | 0.5 | 0.8 | 1.0 | 2.7 |
| pore size | | | | | | | |
| Bubble point | Bar | 0.22 | 0.01 | 1.35 | 0.75 | 0.59 | 0.22 |
| Thickness | μm | 330 | 200 | 28 | 204 | 208 | 204 |
| Base weigh | g/m² | 56 | 70 | 70 | 70 | 70 | 70 |
| membrane | | | | | | | |
| Base weight | g/m² | n.a. | n.a. | 2 | 1 | 2 | 1 |
| nanoweb | | | | | | | |
| Average fiber | μm | n.a. | 40 | 0.12 | 0.17 | 0.27 | 0.32 |
| diameter | | | | | | | |
| Porosity | % | 86 | 70 | 71 | 71 | 71 | 71 |
| membrane | | | | | | | |
| Porosity | % | n.a. | n.a. | 79 | 79 | 79 | 79 |
| nanoweb | | | | | | | |
| Dead volume | μm | 21 | 11 | 12 | 11 | 12 | 11 |
| (Ø = 1 cm) | | | | | | | |
| Mechanical | | compressible | stable | stable | stable | stable | stable |

TABLE 3
Blood separation performance

| | | | | Example 1 | Example 2 | | Example 3 | Example 4 |
|---|---|---|---|---|---|---|---|---|
| | | Comparative | Comparative | Comp B on | Comp B on | Example 2 | Comp B on | Comp B on |
| Features | Unit | Example A | Example B | top | top | NW* top | top | top |
| Blood Volume | μL/cm² | 20 | 10 | 10 | 10 | 10 | 10 | 10 |
| Blood | Sec. | >10 | n.a. | <3 | <3 | N.A. | <3 | <3 |
| Separation | | | | | | | | |
| Time | | | | | | | | |
| Flow pattern | | A | B | B | B | C | B | B |
| Blood | | YES | NO | YES | YES | NO | YES | YES |
| separation | | | | | | | | |
| Generated | μg/1 | 0.13 | 0 | 0.27 | 0.25 | 0 | 0.21 | 0.21 |
| plasma vol. | μL bld | | | | | | | |

A = First vertical then spreading
B = Fast spreading laterally & vertically
C = Pin on deposited spot
*NW stands for nanoweb

1. Membrane construction comprising multiple layers wherein
at least one of the layers is a nanoweb made of polymeric nanofibers
the mean flow pore size of the nanoweb is in the range from 50 nm to 5μm
the number average diameter of the nanofibers is in the range from 100 to 600 nm
the basis weight of the nanoweb is in the range from 1 to 20 g/m²
the porosity of the nanoweb is in the range from 60 to 95%
at least one of the layers is a support layer and
the nanoweb is hydrophilic

2. Membrane construction according to claim 1, wherein the nanofibers are prepared from an aliphatic polyamide, mixtures and copolyamides thereof, preferably polyamide-6, polyamide-6,6, polyamide-4,6, copolyamides and/or mixtures thereof.

3. Membrane construction according to claim 1, wherein the support material is hydrophilic.

4. Membrane construction according to claim 1, wherein the nanofibers are coated, preferably with an antifouling coating.

5. Membrane construction according to claim 1, wherein the antifouling coating is an antibiofouling coating.

6. Membrane construction according to claim 1, wherein the support layer is a microporous layer.

7. Membrane construction according to claim 6, wherein the microporous layer is made from ultra high molecular weight polyethylene, preferably made from a antifouling coated ultra high molecular weight polyethylene.

8. Membrane construction according to claim 1, wherein at least one of the layers of the membrane construction is coated.

9. Membrane construction according to claim 8, wherein at least one of the layers of the membrane construction is coated with an anti(bio)fouling coating.

10. Membrane construction according to claim 1, wherein the support layer is at the top side of the membrane construction and the nanoweb is at the down side of the construction.

11. Membrane cassette comprising the membrane construction of claim 1.

12. Device comprising the membrane construction of claim 1.

13. Use of the membrane construction of claim 1 for blood filtration.

14. Use of the membrane construction of claim 1 for any one of the following applications: molecular separations and filtration, like gas-gas filtration, hot gas filtration, particle filtration, liquid filtration such as micro filtration, ultra filtration, nano filtration, reverse osmosis; waste water purification, oil and fuel filtration; controlled release applications including pharmaceutical and nutraceutical components; pertraction, pervaporation and contactor applications; immobilization of enzymes, and humidifiers, drug delivery; (industrial) wipes, surgical gowns and drapes, wound dressing, tissue engineering, protective clothing, catalyst supports, and various coatings.

15. Use of the membrane construction of claim 1 in a diagnostic device.
